(19) United States
(12) Patent Application Publication
(10) Pub. No.: US 2025/0248674 A1
(43) Pub. Date: Aug. 7, 2025

(54) PARTIAL SCAN AND RECONSTRUCTION FOR A POSITRON EMISSION TOMOGRAPHY SYSTEM

(71) Applicant: Siemens Medical Solutions USA, Inc., Malvern, PA (US)

(72) Inventors: Ziad Burbar, Knoxville, TN (US); Inki Hong, Knoxville, TN (US); Stefan B. Siegel, Knoxville, TN (US)

(21) Appl. No.: 19/186,004

(22) Filed: Apr. 22, 2025

Related U.S. Application Data
(63) Continuation-in-part of application No. 18/906,362, filed on Oct. 4, 2024.
Application No. 18/906,362 is a continuation of application No. 17/597,992, filed on Feb. 1, 2022, now Pat. No. 12,138,096.

(51) Int. Cl. A61B 6/00; A61B 6/03; A61B 6/46

(52) U.S. Cl. CPC A61B 6/5235; A61B 6/032; A61B 6/037; A61B 6/469; A61B 6/5205

(57) ABSTRACT

A system to scan a region of a patient comprises determination of the region, acquisition of PET singles data from a plurality of photosensors, determination of PET singles data which is associated with the region, generation of PET coincidence data based on the PET singles data which is associated with the region, and generation of a PET image based on the PET coincidence data.

### CROSS-REFERENCE TO RELATED APPLICATIONS

This is a continuation-in-part of U.S. patent application Ser. No. 18/906,362, filed Oct. 4, 2024, which is a continuation of U.S. patent application Ser. No. 17/597,992, filed Feb. 1, 2022, which is a 371 application of PCT/US2020/013000, filed Jan. 10, 2020, the contents of each of which are incorporated by reference herein for all purposes.

### BACKGROUND

Nuclear medicine uses radiation emission to acquire images that illustrate the function and physiology of organs, bones or tissues of the body. According to positron-emission-tomography (PET) imaging, a radiopharmaceutical tracer is introduced into a patient body via arterial injection. Radioactive decay of the tracer generates positrons which eventually encounter electrons and are annihilated thereby. The annihilation produces two 511 keV photons which travel in approximately opposite directions.

A ring of detectors surrounding the body detects photons, identifies “coincidences” based thereon, and reconstructs PET images based on the identified coincidences. A coincidence is identified when two photons (i.e., two “singles”) arrive at two detector crystals disposed on opposite sides of the body within a short time window, indicating that the two photons arose from the same positron annihilation. Because the two photons travel in approximately opposite directions, the locations of the two detector crystals determine a line-of-response (LOR) along which an annihilation may have occurred. Time-of-flight (TOF) PET additionally measures the difference between the arrival times of the two photons arising from the annihilation. This difference may be used to estimate a particular position along the LOR at which the annihilation occurred.

PET imaging may be combined with other imaging modalities in a multimodality system. PET-computed tomography (CT) imaging systems allow performance of contemporaneous PET and CT scans in a same coordinate system. The axial field of view (aFoV) of each imaging system is typically similar in order to minimize the impact of patient motion and increase spatial correlation of the respective data sets. PET-CT imaging systems may thereby provide spatially-aligned detailed anatomical and functional information.

Conventional PET-CT imaging systems exhibit an aFoV that matches the size of organs of interest, such as the heart or the brain. To image larger volumes, a patient is scanned at several axial positions relative to the imaging components using either step-and-shoot or continuous bed motion techniques. Long aFoV PET-CT imaging systems attempt to reduce the need for step-and-shoot or continuous bed motion scanning. However, long aFoV CT scans may subject a patient to high doses of radiation and long aFoV PET scans may require significant data storage and processing resources.

Systems are desired to image a region/organ of interest using long aFoV PET-CT imaging systems in a resource- and dose-sensitive manner.

### BRIEF DESCRIPTION OF THE DRAWINGS

The foregoing and other aspects of some embodiments are best understood from the following detailed description when read in connection with the accompanying drawings. Embodiments are not limited to the specific implementations disclosed herein.

FIGS. 1A and 1B illustrate detection of photons by a PET scanner according to some embodiments;

FIG. 2 illustrates detector crystals of PET detectors of a PET scanner according to some embodiments;

FIG. 3 illustrates a PET detector and coincidence detection components of a PET scanner according to some embodiments;

FIGS. 4A and 4B depict detector crystals and corresponding photosensors according to some embodiments;

FIG. 5 is a flow diagram of a process to generate a PET image according to some embodiments;

FIG. 6 is a view of CT imaging components of a PET-CT scanner according to some embodiments;

FIG. 7 illustrates generation of a PET image based on PET data and a linear attenuation coefficient map according to some embodiments;

FIG. 8 illustrates usage of a trained network to generate a PET image based on PET data and a linear attenuation coefficient map according to some embodiments;

FIG. 9 illustrates training of a network to output a linear attenuation coefficient map based on input PET data according to some embodiments;

FIG. 10 is a flow diagram of a process to generate a PET image according to some embodiments; and

FIG. 11 is a block diagram of a PET-CT imaging system according to some embodiments.

### DETAILED DESCRIPTION

The following description is provided to enable any person in the art to make and use the described embodiments. Various modifications, however, will be readily-apparent to those in the art.

Briefly, some embodiments provide efficient and low-dose scanning of a region of interest which has an axial length shorter than the aFoV of the imaging system. In one aspect, PET scintillation pulses are acquired only from PET detector photosensors which are associated with the region of interest and a PET image is reconstructed from the PET scintillation pulses. In another aspect, PET coincidence data is generated based only on PET singles data which is associated with the region of interest while ignoring PET singles data which is not associated with the region of interest, and a PET image is reconstructed based on the PET coincidence data. In yet another aspect, a PET image is reconstructed based only on PET coincidence data which is associated with the region of interest and other PET coincidence data is ignored. Some embodiments thereby reduce the amount of PET singles data and/or PET coincidence data to be stored, transferred and processed. Moreover, embodiments may limit accompanying CT scans to the aFoV of the region of interest, resulting in a reduction of radiation dose to the patient. To further reduce radiation dose, embodiments may reconstruct a PET image based on a linear attenuation coefficient map which itself is generated by inputting the PET data into a trained model.

A region of interest (ROI) may comprise a two-dimensional or three-dimensional region. The ROI may comprise a specific area or volume on which a radiologist, a technician, or software focuses for closer analysis or measurement. For example, analysis may include quantitative analysis to measure metabolic activity in the ROI and measurement may include measurement of size, shape, or volume of a structure. The ROI may be identified in any suitable manner, from manual indication of the ROI on a two- or three-dimensional image to automated image analysis.

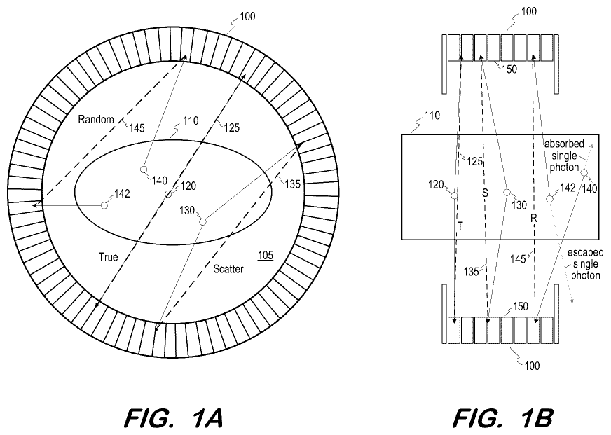

FIG. 1A and FIG. 1B illustrate detection of coincidences by a PET scanner according to some embodiments. FIG. 1A is a transaxial view of bore 105 of PET scanner detector ring 100 and imaging subject 110 disposed therein. Imaging subject 110 may comprise a human body, a phantom, or any other suitable subject. FIG. 1B is an axial view of detector ring 100 and subject 110 of FIG. 1A. Detector ring 100 is composed of an arbitrary number (eight in this example) of adjacent and coaxial rings of detectors 150 in the illustrated example. Each detector 150 may comprise any number of detector crystals and electrical transducers.

The detector crystals may comprise lutetium oxyorthosilicate (LSO), lutetium-yttrium oxyorthosilicate (LYSO), or any other suitable materials that are or become known. According to some embodiments, the electrical transducers may comprise silicon photomultipliers (SiPMs) or photomultiplier tubes (PMTs). The detector crystals create light photons in response to receiving gamma photons. The electrical transducers, or photosensors, convert these light photons to electrical signals, referred to herein as scintillation pulses.

Singles events are determined from the scintillation pulses. An event time is determined for each singles event, representing the time at which its corresponding photon was “detected”. Each singles event is captured as PET singles data indicating a detector crystal at which a photon was received, an event time, and possibly other data (e.g., pulse energy, pulse amplitude).

The determination of singles events includes rejecting pulses from photons with energies below ˜511 keV and determining a detector crystal corresponding to each received (and energy-qualified) photon. In some implementations, each detector crystal is optically coupled to one specific SiPM transducer so all pulses generated by the transducer are assumed to have been caused by a photon received at its corresponding detector crystal. According to other implementations, more than one electrical transducer may receive light generated by a detector crystal. A weighted sum of the corresponding scintillation pulses may be used to estimate an interaction position, and the crystal closest to the estimated position is determined to correspond to the received photon. According to light-sharing techniques, in which scintillation light is also spread over multiple detectors, a pre-computed look-up table maps pulse patterns to specific detector crystals.

Annihilations 120, 130, 140 and 142 are assumed to occur at various locations within subject 110. As described above, an injected tracer generates positrons which are annihilated by electrons to produce two 511 keV photons which travel in approximately opposite directions. Each of annihilations 120, 130, 140 and 142 results in the detection of a coincidence. True coincidences represent valid image data, while scatter and random coincidences represent noise associated with incorrect event position information.

Coincidences are detected based on PET singles data. A coincidence is detected when two singles event times are within a specified coincidence time window of one another. For example, annihilation 120 resulted in two photons which were detected within the coincidence time window. These detections represent a true coincidence because the position of annihilation 120 lies on LOR 125 which connects the positions of the detector crystals at which the two photons were received.

Annihilation 130 results in scatter coincidence because, even though the two photons resulting from annihilation 130 were detected within the coincidence time window, the position of annihilation 130 does not lie on LOR 135 connecting the two crystals which received the photons. This may be due to Compton (i.e., inelastic) or Coherent (i.e., elastic) scatter resulting in a change of direction of at least one of the two photons within subject 110.

Annihilations 140 and 142 are two separate annihilations which result in detection of a random coincidence. In the present example, one of the photons generated by annihilation 140 is absorbed in body 210 and one of the photons generated by annihilation 142 escapes detection by any detector 150 of detector ring 100. The remaining two photons generated by the two annihilations happen to be detected within the coincidence time window, even though no annihilation occurred on LOR 145 connecting the positions at which the coincident photons were received.

The detected coincidences may be stored as PET coincidence data comprising raw (i.e., list-mode) data and/or sinograms. List-mode data may represent each coincidence via data identifying the two detector crystals which define the LOR of the coincidence and the event times of the scintillation pulses of the coincidence. Since only the true unscattered coincidences indicate locations of actual annihilations, random coincidences and scatter coincidences are often subtracted from or otherwise used to correct the PET coincidence data prior to or during reconstruction of a PET image based thereon.

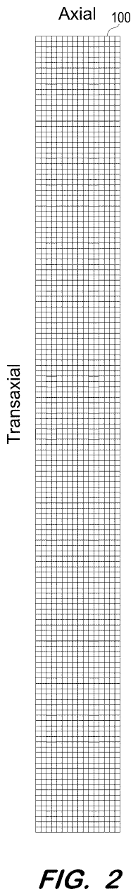

FIG. 2 presents detector ring 100 in an unrolled configuration. Each square represents a “mini-block” of detector crystals according to some embodiments. Ring 100 includes 16 mini-blocks in the axial direction and 152 mini-blocks in the transaxial direction. In one example, each mini-block comprises a grid of 5×5 LSO crystals having dimensions of 3.2 mm×3.2 mm×20 mm.

According to non-exhaustive embodiments, a “detector” is composed of eight mini-blocks, with two mini-blocks in the axial direction and four mini-blocks in the transaxial direction. With reference to the mini-block configuration described above, a detector includes 200 crystals, with rows of 10 crystals in the axial direction and 20 crystals in the transaxial direction. Ring 100 includes eight detectors in the axial direction and thirty-eight detectors in the transaxial direction. Ring 100 therefore includes 60800 detector crystals, with rows of 80 detector crystals in the axial direction and rows of 760 detector crystals in the transaxial direction. Embodiments are not limited to the specific structure or components of detector ring 100.

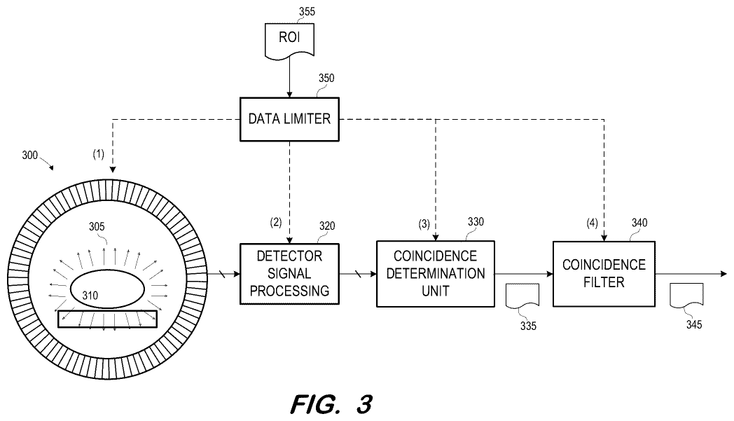

FIG. 3 illustrates PET detector ring 300 of a PET scanner according to some embodiments. Detector ring 300 includes a plurality of detectors in the axial direction as well as the illustrated detectors in the transaxial direction. Detector ring 300 receives photons 305 emitted from volume 310. As described above, the detector crystals of detector ring 300 receive the photons 305 and emit light photons in response. Corresponding photosensors receive the light photons from the detector crystals and each photosensor generates electrical signals (i.e., scintillation pulses) based on the energy of the light photons it receives and its own characteristic photoelectric response profile.

Detector signal processing unit 320 is configured to receive scintillation pulses from photosensors 310, to reject invalid (e.g., low-energy) pulses, and generate PET singles data indicating a detector crystal and an event time for each valid received photon as described above. Coincidence determination unit 330 is configured to receive PET singles data from detector signal processing unit 320. Coincidence determination unit 330 is capable of identifying a coincidence event for each pair of singles events whose event times fall within a coincidence time window.

Coincidence determination unit 330 outputs PET coincidence data 335, which may comprise list-mode data, sinogram data, etc. Coincidence filter 340 may filter PET coincidence data 335 based on specified criteria, resulting in filtered PET coincidence data 345. Detector signal processing unit 320, coincidence determination unit 330 and coincidence filter 340 may comprise any hardware and/or software may perform any suitable functions and exhibit any suitable implementations.

FIG. 3 illustrates four different techniques for limiting data processing based on ROI data 355. Any one or more of the four techniques may be implemented within a system according to some embodiments. Data 355 may specify locations of one or more regions to be imaged within the bore of the PET scanner. Data 355 may define the ROI in terms of LORs which pass through the one or more regions, detector crystals which comprise those LORs, detector transducers, detector blocks, detector mini-blocks, and/or any suitable structures.

Data limiter 350 may issue commands to control (1) photosensors of detector ring 300, (2) detector signal processing unit 320, (3) coincidence determination unit 330 and/or (4) coincidence filter 340 based on ROI data 355. For example, data limiter 350 may determine and disable one or more photosensors of ring 300 which are not associated with the one or more regions. A photosensor may be determined to be associated with the one or more regions if it is configured to receive light from a detector crystal which defines (in conjunction with another crystal of the detector ring) at least one LOR which passes through the one or more regions. Disabling a photosensor may comprise disabling power to the photosensor or blocking transmission of a scintillation pulse from the photosensor to detector signal processing unit 320, for example. Since photosensors which are not associated with the one or more regions are disabled in this example, detector signal processing unit 320 will receive scintillation pulses only from photosensors which are associated with the one or more regions.

According to another technique, data limiter 350 may instruct detector signal processing unit 320 to block certain PET singles data from reaching coincidence determination unit 330. In other words, data limiter 350 may control detector signal processing unit 320 to pass only PET singles data which is associated with the one or more regions of interest to coincidence determination unit 330. The PET singles data to block/pass may be determined based on the detector crystals with which the PET singles data is associated. For example, PET singles data associated with crystals belonging to LORs which pass through the region of interest (or crystals of designated detector blocks, mini-blocks, etc.) may be passed to coincidence determination unit 330, while all other PET singles data is blocked.

Data limiter 350 may also or alternatively instruct coincidence determination unit 330 to ignore PET singles data received from detector signal processing unit 320 and unrelated to the ROI when identifying coincidence events. Unit 330 may be instructed to ignore PET singles data which is not associated with crystals belonging to LORs which pass through the region of interest (or crystals of ROI-associated detector blocks, mini-blocks, for example).

In yet another possibility, data limiter 350 instructs coincidence filter 340 to discard PET coincidence data 335 which is not associated with LORs which pass through the region of interest (or crystals thereof). Consequently, PET coincidence data 345 consists only of coincidences associated with LORs which pass through the region of interest.

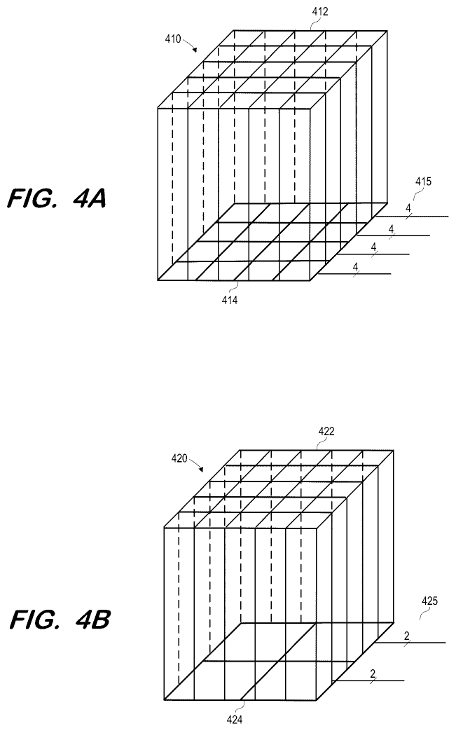

FIGS. 4A and 4B illustrate mini-blocks of a detector ring according to some embodiments. Each of mini-blocks 410 and 420 comprises a grid of 5×5 LSO crystals having dimensions of 3.2 mm×3.2 mm×20 mm. Crystals 412 of mini-block 410 are coupled to a 4×4 array of photosensors 414 (e.g., SiPM devices) for receiving light photons therefrom and generating scintillation pulses based thereon and crystals 422 mini-block 420 are coupled to a 2×2 array of such photosensors 424.

Referring to FIG. 4A, each one of sixteen signal lines 415 is electrically connected to a respective sixteen of four photosensors 414 and to detector signal processing unit 320. Blocking one of signal lines 415 prevents scintillation pulses generated by the photosensor 414 which is coupled to the blocked signal line 415 from reaching detector signal processing unit 320. As a result, light photons received by the photosensor 414 from adjacent ones of crystals 412 are ignored during subsequent processing.

Each one of sixteen signal lines 425 of FIG. 4B is electrically connected to a respective one of four photosensors 424 and to detector signal processing unit 320. A gain, blocking one of signal lines 425 prevents scintillation pulses generated by the corresponding photosensor 424 from reaching detector signal processing unit 320. The greater number of photosensors per crystal in FIG. 4B allows for finer control over which detector crystals are used to detect photons from which image data will be generated.

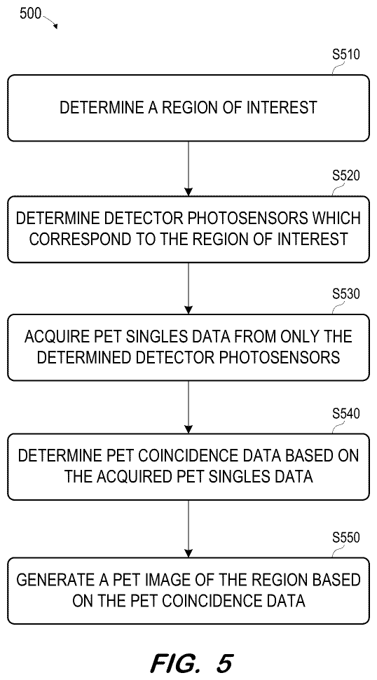

FIG. 5 is a flow diagram of process 500 to generate a PET image according to some embodiments. Process 500 may be performed by any combination of hardware and software that is or becomes known. Program code embodying these processes may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processing cores, and processor threads. Embodiments are not limited to the examples described below.

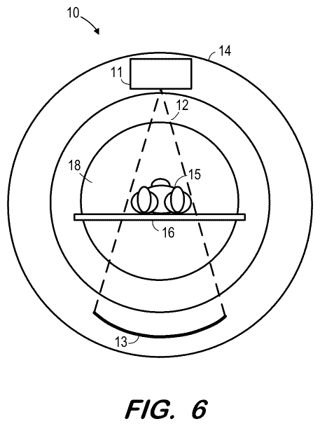

A region of interest is determined at S510. S510 may include various techniques to allow an operator to select the region of interest. For example, S510 may include acquisition of a CT topogram of a patient's body using the CT imaging components of a PET-CT system. FIG. 6 shows CT imaging system 10 according to some embodiments. CT imaging system 10 comprises X-ray source 11 for emitting X-ray beam 12 toward opposing radiation detector 13. X-ray source 11 and radiation detector 13 are mounted on gantry 14 such that they may be rotated about a center of rotation of gantry 14 while maintaining the same physical relationship therebetween during acquisition of CT data.

To generate a CT topogram, X-ray source 11 and radiation detector 13 remain in a fixed position while bed 16 is moved axially so that the entire length of patient 15 passes between source 11 and detector 13. During this movement, X-ray source 11 is powered to transmit X-ray radiation 12 toward detector 13. Detector 13 receives the radiation and a full-length X-ray image of patient 15 (i.e., a topogram) is generated therefrom.

The topogram may be displayed to an operator, and the operator may select the region of interest from the displayed topogram. In some embodiments, a series of blocks extending axially along at least one side of the topogram are presented on an operator interface. Each block in the series corresponds to a portion of the topogram. Operator selection of the region of interest comprises a selection of one or more of the blocks (e.g., via clicking on the block(s) of interest). In other embodiments, operator selection of the region of interest comprises drawing of one or more shapes on one or more portions of the topogram. Determination of the region of interest may be performed automatically using image processing techniques or machine learning. For example, based on the type of study being performed, a machine learning model may analyze an image to locate an organ or tissues of interest and the surrounding area that is needed to perform the desired study.

Returning to process 500, photosensors which correspond to the region of interest are determined at S520. In some embodiments, a subset of PET detectors, mini-blocks, or detector crystals that are outside the region of interest are identified, resulting in a different subset of PET detectors, mini-blocks, or detector crystals that correspond to the region of interest. This identification may be automatically performed by the PET-CT system or manually performed by an operator. S520 may include determining LORs which pass through (or which do not pass through) the region of interest, and in particular the pairs of detector crystals which define the LORs.

In some embodiments, determination of the region of interest comprises identification of an axial region of interest in a coordinate space of the PET-CT system. The identified axial region of interest is represented by radial offset values and a restricted ring difference value, where the restriction is based on LORs which traverse the region of interest. Accordingly, S520 may include determination of detectors which satisfy the radial offset values and the restricted ring difference value. Once the detectors, mini-blocks, or detector crystals which correspond to the region of interest are identified, the corresponding photosensors may also be determined based on the structure of the PET detectors.

At S530, a PET scan is performed in which PET singles data is acquired from only the detector photosensors which have been determined to correspond to the region of interest. Acquisition of PET singles data from only those detector photosensors may comprise disabling the detector photosensors which do not correspond to the region of interest. Alternatively, as described above, PET singles data generated by detector photosensors which do not correspond to the region of interest may be blocked from reaching a coincidence determination unit.

PET coincidence data is determined at S540 based on the PET singles data acquired at S530. S540 comprises identifying a coincidence for each pair of the acquired PET singles data whose event times fall within a coincidence time window. A determined coincidence may be represented by the two detector crystals associated with the pair of PET singles data, and a difference between their respective event times.

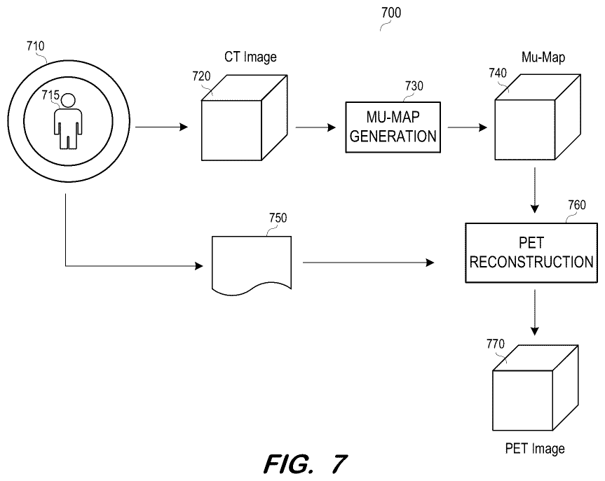

A PET image of the region of interest is generated at S550 from the determined PET coincidence data. FIG. 7 illustrates PET image generation according to some embodiments. PET-CT scanner 710 acquires CT data of patient 715 and applies conventional CT reconstruction algorithms to the acquired CT data to generate CT image 720. M u-map generation component 730 derives linear attenuation coefficient map (i.e., “mu-map”) 740 from CT image 720 as is known in the art. A mu-map provides attenuation coefficients of subject tissue and is typically used for attenuation correction of PET data during image reconstruction.

Scanner 710 acquires PET coincidence data 750 as described with respect to process 500. PET coincidence data 750 comprises the coincidences determined at S540 based only on PET singles data generated by photosensors corresponding to the region of interest. PET coincidence data 750 may represent the detected coincidences as raw (i.e., list-mode) data and/or sinograms. List-mode data represents each coincidence using data specifying a LOR between two detector crystals, the time at which each photon of the coincidence reached each crystal, the photon energies, etc. A sinogram is a data array of the angle versus the displacement of the LORs of each detected coincidence. A sinogram includes one row containing the LOR for a particular azimuthal angle q.

PET reconstruction component 760 reconstructs three-dimensional PET image 770 based on mu-map 740 and PET data 750. As is known in the art. PET reconstruction component 760 may reconstruct PET image 770 using algorithms such as filtered backprojection (FBP) and ordered subsets expectation maximization (OSEM), but embodiments are not limited thereto. Reconstruction may include any other suitable steps, such as subtraction of random coincidences and scatter coincidences from PET data 750, motion correction, and correction for system sensitivity.

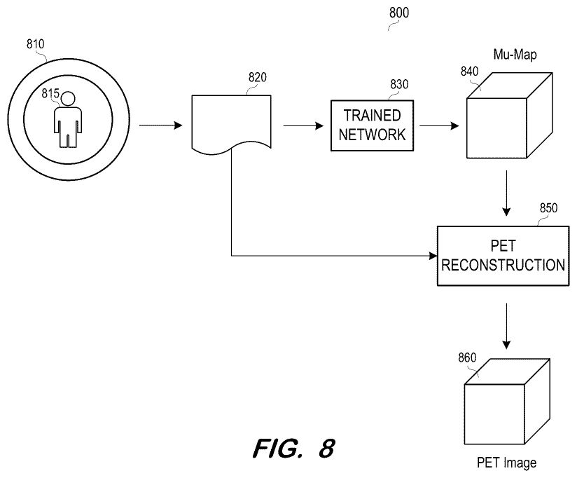

FIG. 8 is a block diagram of system 800 to generate a PET image at S540 based on a network-generated mu-map according to some embodiments. PET-CT scanner 810 acquires PET coincidence data 820 of patient 815 as described with respect to process 500. PET data 820 is input to trained neural network 830 to generate mu-map 840. M u-map 840 is intended to simulate a mu-map determined from a CT image of patient 815. PET reconstruction component 850 uses mu-map 840 and PET data 820 to reconstruct PET image 860 as described above. While system 800 may employ a CT topogram to determine the region of interest as described above, system 800 advantageously avoids performance of a CT scan and the accompanying radiation exposure to the patient.

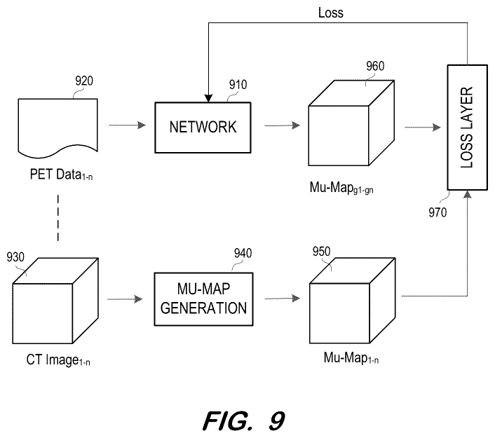

FIG. 9 illustrates training of neural network 910 to generate a mu-map based on PET data according to some embodiments. Network 910 may comprise any type of suitable neural network that is or becomes known. FIG. 9 depicts N sets of PET coincidence data 920 and N corresponding (e.g., contemporaneously-acquired) CT images 930. M u-map generation component 940 generates N mu-maps 950 from corresponding ones of N photon-counting CT images 930. Accordingly, each of the N mu-maps 950 is a “ground truth” associated with a corresponding one of the N sets of PET data 920.

In one example of network training, a batch of M PET data 920 is input to network 910. Network 910 generates a simulated mu-map 960 from each PET data 920 of the batch. Loss layer 970 calculates a loss based on differences between each of the M generated simulated mu-maps 960 and a corresponding ground truth mu-map 950. The loss is back-propagated to network 910 and the process repeats until training is complete. The resulting trained network 910 may be deployed in a system such as system 800 to generate simulated mu-maps from input PET data.

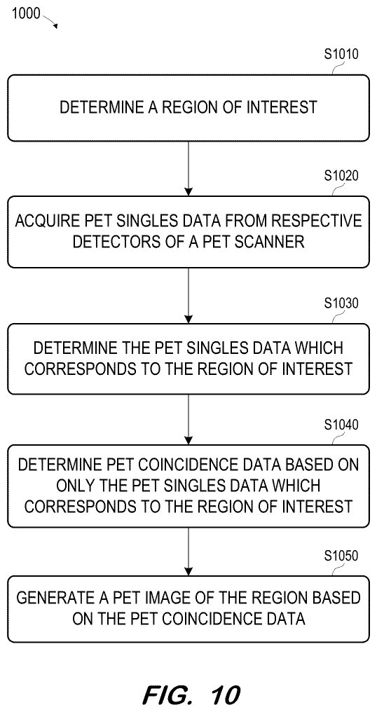

FIG. 10 is a flow diagram of process 1000 to generate a PET image according to some embodiments. A region of interest is determined at S1010 using any of the techniques described above or other techniques. Next, at S1020, a PET scan is performed to acquire PET singles data from the photosensors of a PET scanner. In contrast to S530, PET singles data is acquired at S1020 from detector photosensors which correspond to the region of interest and those which do not correspond to the region of interest.

At S1030, PET singles data which corresponds to the region of interest is determined. Determination of the PET singles data which corresponds to the region of interest may proceed as described above with respect to S520. PET coincidence data is determined at S1040 based only on the PET singles data which corresponds to the region of interest. As described above with respect to FIG. 3, a coincidence determination unit which receives all the PET singles data acquired at S1020 may be instructed to consider only the PET singles data which corresponds to the region of interest when identifying pairs of PET singles data whose event times fall within the coincidence time window. Limiting the number of considered s PET singles data may reduce the processing resources and time required to identify the coincidences at S1040. Next, at S1050, a PET image of the region of interest is generated from the determined PET coincidence data, for example as illustrated in FIG. 7 or 8.

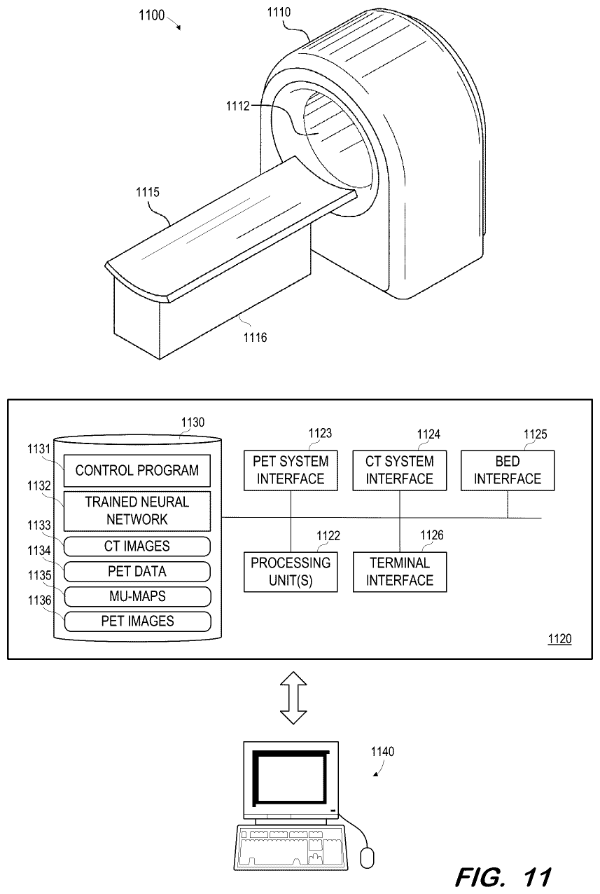

FIG. 11 illustrates PET-CT scanner 1100 to execute one or more of the processes described herein. Embodiments are not limited to scanner 1100 or to a multi-modality imaging system.

Scanner 1100 includes gantry 1110 defining bore 1112. As is known in the art, gantry 1110 houses PET imaging components for acquiring PET image data and CT imaging components for acquiring CT image data. The CT imaging components may include one or more x-ray tubes and one or more corresponding X-ray detectors as is known in the art. The PET imaging components may include any number or type of detectors disposed in any configuration as is known in the art.

Bed 1115 and base 1116 are operable to move a patient lying on bed 1115 into and out of bore 1112 before, during and after imaging. In some embodiments, bed 1115 is configured to translate over base 1116 and, in other embodiments, base 1116 is movable along with or alternatively from bed 1115.

Movement of a patient into and out of bore 1112 may allow scanning of the patient using the CT imaging elements and the PET imaging elements of gantry 1110. Bed 1115 and base 1116 may provide continuous bed motion and/or step-and-shoot motion during such scanning according to some embodiments.

Control system 1120 may comprise any general-purpose or dedicated computing system. Accordingly, control system 1120 includes one or more processing units 1122 configured to execute program code to cause system 1120 to acquire image data and generate images therefrom, and storage device 1130 for storing the program code. Storage device 1130 may comprise one or more fixed disks, solid-state random-access memory, and/or removable media (e.g., a thumb drive) mounted in a corresponding interface (e.g., a Universal Serial Bus port).

Storage device 1130 stores program code of control program 1131. One or more processing units 1122 may execute control program 1131 to control CT imaging elements of scanner 1100 using CT system interface 1124 and bed interface 1125 to acquire CT data and to reconstruct CT images 1133 therefrom. One or more processing units 1122 may execute control program 1131 to, in conjunction with PET system interface 1123 and bed interface 1125, control hardware elements to inject a radiopharmaceutical into a patient, move the patient into bore 1112 past PET detectors of gantry 1110, and acquire PET data 1134 as described above.

Trained neural network 1132 may be executable to generate mu-map 1135 from PET data 1134. Such a mu-map 1135 may be used to reconstruct a PET image 1136 from PET data 1134. PET images 1136 and CT images 1133 may be transmitted to terminal 1140 via terminal interface 1126. Terminal 1140 may comprise a display device and an input device coupled to system 1120. Terminal 1140 may display the received PET images 1136 and CT images 1134. Terminal 1140 may receive operator input for selecting a region of interest, controlling display of the data, operation of scanner 1100, and/or the processing described herein. In some embodiments, terminal 1140 is a separate computing device such as, but not limited to, a desktop computer, a laptop computer, a tablet computer, and a smartphone.

Each component of scanner 1100 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

What is claimed is:

1. A system comprising:
a plurality of positron emission tomography (PET) detectors, each of the PET detectors comprising a plurality of photosensors and one or more detector crystals associated with each photosensor,
the system configured to:
determine a region of interest to be scanned;
acquire scintillation pulses from only ones of the plurality of photosensors which are associated with the region;
generate PET coincidence data based on the scintillation pulses; and
generate a PET image based on the PET coincidence data.

2. The system of claim 1, wherein generation of the PET coincidence data based on the scintillation pulses comprises determination of coincidences based on the scintillation pulses, and wherein generation of the PET image is based on the coincidences.

3. The system of claim 1, wherein acquisition of the scintillation pulses from only ones of the plurality of photosensors which are associated with the region comprises determination of the ones of the plurality of photosensors which are associated with the region and disabling other ones of the plurality of photosensors.

4. The system of claim 3, wherein determination of the ones of the plurality of photosensors which are associated with the region comprises determination of ones of the detector crystals which are associated with the region, and determination of photosensors which are associated with determined detector crystals.

5. The system of claim 4, wherein determination of the ones of the detector crystals which are associated with the region comprises determination of lines-of-response which are associated with the region and determination of detector crystals which are associated with the lines-of-response.

6. The system of claim 3, wherein determination of the ones of the plurality of photosensors which are associated with the region is based on radial offset values and a restricted ring difference value corresponding to the region.

7. The system of claim 1, wherein generation of the PET image comprises input of the PET coincidence data to a trained network to generate a linear attenuation coefficient map, and generation of the PET image based on the PET coincidence data and the linear attenuation coefficient map.

8. A system comprising:
a plurality of positron emission tomography (PET) detectors, each of the PET detectors comprising a plurality of photosensors and one or more detector crystals associated with each photosensor,
the system configured to:
determine a region of interest to be scanned;
acquire scintillation pulses from the plurality of photosensors;
determine PET singles data from the acquire scintillation pulses;
generate PET coincidence data based only on the PET singles data which is associated with the region; and
generate a PET image based on the PET coincidence data.

9. The system of claim 8, further comprising a coincidence determination unit,
wherein the system is configured to transmit determined PET singles data which is associated with the region to the coincidence determination unit and to not transmit determined PET singles data which is not associated with the region to the coincidence determination unit.

10. The system of claim 8, further comprising a coincidence determination unit to receive the determined PET singles data and to determine coincidences based on the determined PET singles data which is associated with the region and not on the determined PET singles data which is not associated with the region.

11. The system of claim 8, wherein acquisition of the scintillation pulses from the plurality of photosensors comprises determination of ones of the plurality of photosensors which are not associated with the region and disabling the determined ones of the plurality of photosensors.

12. The system of claim 11, wherein determination of the ones of the plurality of photosensors which are not associated with the region comprises determination of ones of the detector crystals which are not associated with the region, and determination of photosensors which are associated with determined detector crystals.

13. The system of claim 12, wherein determination of the ones of the detector crystals which are not associated with the region comprises determination of lines-of-response which are not associated with the region and determination of detector crystals which are associated with the lines-of-response.

14. The system of claim 11, wherein determination of the ones of the plurality of photosensors which are not associated with the region is based on radial offset values and a restricted ring difference value corresponding to the region.

15. The system of claim 8, wherein generation of the PET image comprises input of the PET coincidence data to a trained network to generate a linear attenuation coefficient map, and generation of the PET image based on the PET coincidence data and the linear attenuation coefficient map.

16. A method to generate a positron emission tomography (PET) image using a plurality of PET detectors, each of the PET detectors comprising a plurality of photosensors and one or more detector crystals associated with each photosensor, the method comprising:
determining a region of a patient;
acquiring PET singles data from only ones of the plurality of photosensors which are associated with the region;
generating PET data based on the scintillation pulses; and
generating a PET image based on the PET data.

17. The method of claim 16, wherein acquiring the scintillation pulses from only ones of the plurality of photosensors which are associated with the region comprises determining the ones of the plurality of photosensors which are associated with the region and disabling other ones of the plurality of photosensors.

18. The method of claim 17, wherein determining the ones of the plurality of photosensors which are associated with the region comprises:
determining lines-of-response which are associated with the region;
determining ones of the detector crystals which are associated with the lines-of-response; and
determining the ones of the plurality of photosensors which are associated with the determined ones of the detector crystals.

19. The method of claim 17, wherein determining the ones of the plurality of photosensors comprises:
determining radial offset values and a restricted ring difference associated with the region;
determining ones of the detector crystals which satisfy the radial offset values and the restricted ring difference; and
determining the ones of the plurality of photosensors which are associated with the determined ones of the detector crystals.

20. A method to generate a positron emission tomography (PET) image using a plurality of PET detectors, each of the PET detectors comprising a plurality of photosensors and one or more detector crystals associated with each photosensor, the method comprising:
determining a region of a patient;
acquire scintillation pulses from the plurality of photosensors;
determine PET coincidence data based on the scintillation pulses;
identify the PET coincidence data which is associated with the region;
output the PET coincidence data which is associated with the region and not the PET coincidence data which is not associated with the region; and
generate a PET image based on the output PET coincidence data which is associated with the region.
